(19) United States
(12) Patent Application Publication
(10) Pub. No.: US 2019/0082934 A1
(43) Pub. Date: Mar. 21, 2019

(54) FLEXIBLE TUBE FOR ENDOSCOPE

(71) Applicant: Olympus Corporation, Tokyo (JP)

(72) Inventor: Takashi Matsunaga, Tokyo (JP)

(73) Assignee: Olympus Corporation, Tokyo (JP)

(21) Appl. No.: 16/192,044

(22) Filed: Nov. 15, 2018

Related U.S. Application Data
(63) Continuation of application No. PCT/JP2017/005461, filed on Feb. 15, 2017.

(30) Foreign Application Priority Data
May 31, 2016 (JP) 2016-109023

(51) Int. Cl. A61B 1/005; A61B 1/01; G02B 23/24; A61B 1/06; A61B 1/00

(52) U.S. Cl. CPC A61B 1/0051; A61B 1/01; A61B 1/00078; A61B 1/06; G02B 23/2476

(57) ABSTRACT

An endoscope includes a tube, a first rigid portion, a second rigid portion, a bent shape maintaining member, and a biasing member. The tube has respective opposed first and second ends. The first rigid portion is attached to the first end. The second rigid portion is attached to the second end. The bent shape maintaining member has respective opposed third and fourth ends and is disposed in the tube and is able to be bent relative to a direction of a longitudinal axis of the bent shape maintaining member and is maintained in a bent shape with the third end being attached to the first rigid portion and the fourth end being slidably disposed at the second rigid portion. The biasing member is disposed at the second rigid portion that biases the fourth end along the longitudinal axis toward the third end.

### CROSS-REFERENCE TO RELATED APPLICATIONS

This application is a continuation application of PCT Application No. PCT/JP2017/005461 filed on Feb. 15, 2017, which in turn claim priority to the Japanese Patent Application No. 2016-109023 filed on May 31, 2016 in Japan which is hereby incorporated by reference in its entirety.

### TECHNICAL FIELD

The technology disclosed herein generally relates to a flexible tube for an endoscope that includes a bent posture holding portion.

### DESCRIPTION OF THE RELATED ART

Endoscopes are used for observation, treatment and the like of the inside of a living body, or for inspection, repair and the like in industrial plant equipment. The endoscopes for medical use may be endoscopes of which an insertion portion is flexible and which are inserted into the upper gastrointestinal tract, the lower gastrointestinal tract or the like, or endoscopes of which an insertion portion is rigid and which are inserted into the abdominal cavity, the thoracic cavity or the like.

In a surgical endoscope device of Japanese Patent No. 5865559, there is depicted an endoscope that includes an insertion portion. The insertion portion includes a metallic distal portion, a freely bendable portion, and a metallic rigid pipe or tube. A bent posture holding portion is disposed at a proximal end of the insertion portion. This endoscope enables the user, such as an endoscopist, to grip and operate the endoscope in a state in which the visual field direction of the endoscope is stabilized, without taking an unreasonable posture.

The bent posture holding portion is a flexible tube, and includes mainly of a bent posture holding pipe and an over-tube or an outside flexible tube. The bent posture holding pipe is freely bendable and is able to be attachably held while maintaining an arbitrarily bent state. The over-tube covers the bent posture holding pipe. The bent posture holding pipe includes a first spiral pipe formed by spirally winding a wire material, and a second spiral pipe formed by winding a wire material having a triangular sectional shape around the first spiral pipe.

The bent posture holding portion depicted in Japanese Patent No. 5865559 is repeatedly bent by the user. Due to the repeated bending, the first spiral pipe and the second spiral pipe rub against each other and thus are eventually worn. Then, the overall length of the bent posture holding pipe is gradually shortened as compared to the over-tube constituting the bent posture holding portion. As a result, a loosened portion is generated in the over-tube, resulting in that when the user makes a bending operation of the bent posture holding portion, the torque exerted on the bent posture holding pipe increases according to loosening of the over-tube. In addition, a partial load is exerted on the loosened portion of the over-tube, leading to deterioration or breakage of the tube, so that the function as the bent posture holding portion may be spoiled.

### BRIEF SUMMARY OF EMBODIMENTS

It is therefore an object of the present disclosure to provide a flexible tube for an endoscope by which the problem of spoiling of the function of a bent posture holding portion due to shortening of the overall length of a bent posture holding pipe is being overcomed.

A flexible tube for an endoscope according to an aspect of the present disclosure includes a first rigid portion, a second rigid portion, a torsion preventing member, a bent shape maintaining member, and a biasing member. The first rigid portion constitutes a first end portion which is on one end side of the flexible tube. The second rigid portion constitutes a second end portion which is an end portion of the flexible tube on an opposite side of the first end portion. The torsion preventing member is formed in an elongated thin pipe shape from a flexible member. The torsion preventing member has a third end portion and a fourth end portion. The third end portion is on the one end side attached to the first rigid portion. The fourth end portion is an end portion on an opposite side of the third end portion. The fourth end portion is connected or attached to the second rigid portion. The torsion preventing member constitutes an exterior part. The bent shape maintaining member is formed in a pipe shape from a metallic member such as to be bendable relative to a direction of a longitudinal axis thereof and to be able to maintain a bent shape. The bent shape maintaining member is disposed in a pipe of the torsion preventing member. The bent shape maintaining member has a fifth end portion on the one end side and a sixth end portion as an end portion on an opposite side of the fifth end portion. One of the fifth end portion and the sixth end portion is attached to in either a first hole or a second hole. The first hole is located in the first rigid portion. The second hole is located in the second rigid portion. The other of the fifth end portion and the sixth end portion is slidably disposed in the first hole or the second hole. The biasing member is disposed in the first hole or the second hole. The biasing member biases the end portion on one side of the bent shape maintaining member slidably disposed in the first hole or the second hole along the longitudinal axis of the bent shape maintaining member toward the end portion on an other side of the bent shape maintaining member.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a side perspective view of a flexible tube of a rigid endoscope described herein.

FIG. 2 is a cross-sectional view of a bent posture holding portion as the flexible tube taken along line 2-2 in FIG. 1.

FIG. 3A is an enlarged view of a portion of FIG. 2 illustrating an over-tube as a torsion preventing member.

FIG. 3B is an enlarged view of a portion of FIG. 2 illustrating a distal-side rigid portion constituting a first end portion of the bent posture holding portion.

FIG. 3C is an enlarged view of a portion of FIG. 2 illustrating a proximal-side rigid portion constituting a second end portion of the bent posture holding portion.

FIG. 3D is an enlarged view of a portion of FIG. 2 illustrating the configuration of a bent posture holding pipe.

FIG. 3E is an enlarged view of a portion of FIG. 2 illustrating a sliding tube attached to an end portion of the bent posture holding pipe.

FIG. 3F is an enlarged view of a portion of FIG. 2 illustrating a push spring as a biasing member.

FIG. 4 is an exploded view illustrating the relations between the bent posture holding pipe and the push spring and the distal-side rigid portion and the proximal-side rigid portion.

FIG. 5 is an exploded view illustrating the relations between the over-tube, which is attached to the distal-side rigid portion and the proximal-side rigid portion, and the bent posture holding pipe and the push spring.

FIG. 6 is a cross-sectional view of the bent posture holding portion in an assembled state.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

In the drawings to be used for the following description, the contraction scale may be different from component to component, for ensuring that each component is in such a size as to be recognizable on the drawings. In other words, the present invention is not limited only to the numbers or amounts of the components described in the drawings, the shapes of the components, dimensional ratios of the components, and relative positional relations of the components.

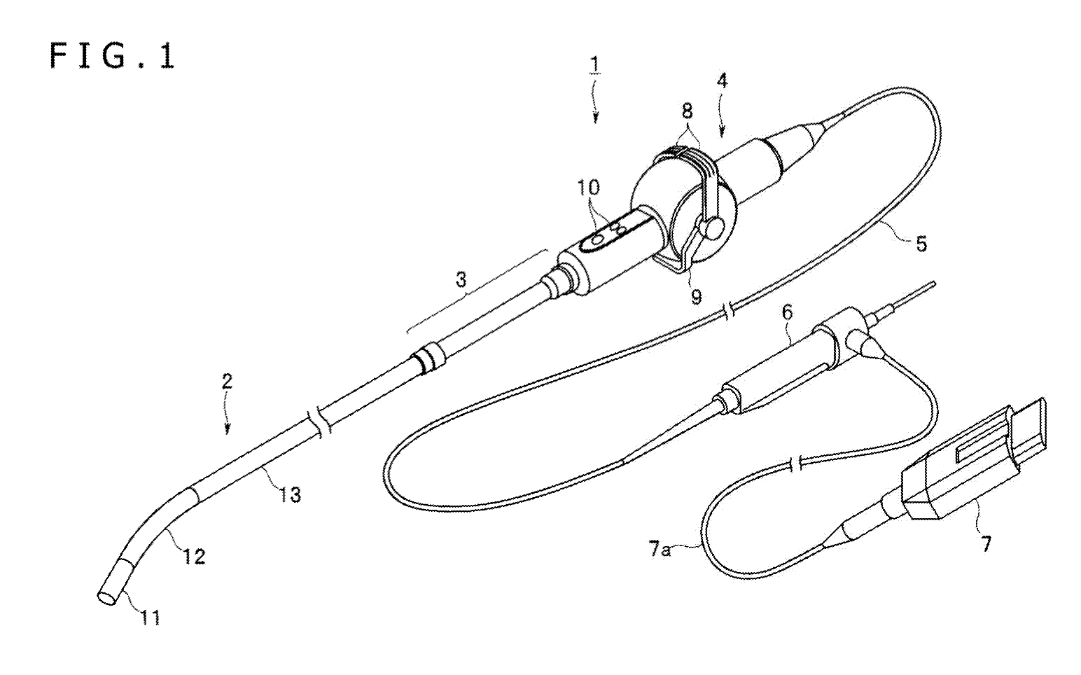

An endoscope 1 depicted in FIG. 1 is one of rigid endoscopes for use in endoscopic surgery or the like. The endoscope 1 includes, on the proximal side of an insertion portion 2, a bent posture holding portion 3 which is a flexible tube for an endoscope. In addition, an operation portion 4 is disposed on the proximal side of the bent posture holding portion 3. A universal cord 5 is extended from the operation portion 4. A light guide connector 6 is connected to a light source device, not depicted, that is as an external apparatus. A video connector 7 is connected to a video system center, not depicted, that is an external apparatus. An electric cable 7a has an extension end of the cable extended from the video connector 7 connected to the light guide connector 6.

The insertion portion 2 includes a distal portion 11, a bendable portion 12, and a rigid pipe 13. The distal portion 11 is formed of a metallic member of stainless steel or the like. The bendable portion 12 is configured to be bent into upward, downward, leftward, and rightward directions. The rigid pipe 13 is formed of a metallic tube or pipe member of stainless steel or the like. The distal portion 11 contains an imaging unit, an illumination lens unit and the like, which are not illustrated. The bendable portion 12 includes a bending piece assembly or an endoscope bendable pipe or tube. A plurality of bending pieces are rotatably connected in the bending piece assembly. The endoscope bendable pipe is formed by including a rigid pipe with a plurality of slots. A bending wire corresponding to upward, downward, leftward, and rightward directions is inserted in and passed through the bendable portion 12.

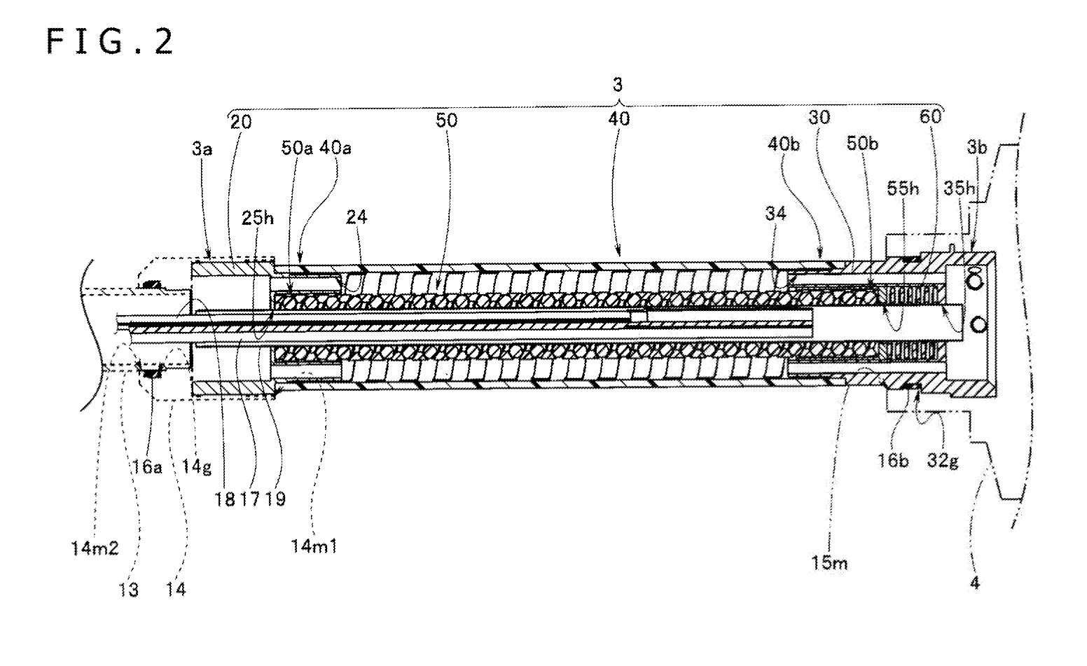

The operation portion 4 includes two angle levers 8, an engagement lever 9, and various switches 10. The angle levers 8 are levers for a bending operation of the bendable portion 12, and pulls and loosens the bending wire. The engagement lever 9 is a lever which is operated at the time of maintaining a bent state of the bendable portion 12 or at the time of releasing the maintained state. The various switches 10 are switches for instructing stopping or recording of an endoscopic image displayed on a screen of a display device, not depicted, enlargement of the image, switching of illumination light, etc. Note that the bending directions of the bendable portion 12 are not limited to the four directions, namely, the upward, downward, leftward, and rightward directions. They may be two directions, specifically, the upward and downward directions, or the leftward and rightward directions. Hereinafter, the bent posture holding portion 3 will be described referring to FIGS. 2 to 6.

As depicted in FIG. 2, the bent posture holding portion 3 is disposed between the insertion portion 2 and the operation portion 4, and is set to a predetermined length. The bent posture holding portion 3 includes mainly a distal-side rigid portion 20, a proximal-side rigid portion 30, an over-tube 40, a bent posture holding pipe 50, and a biasing member. The distal-side rigid portion 20 is a first rigid portion. The proximal-side rigid portion 30 is a second rigid portion. The over-tube 40 is a torsion preventing member. The bent posture holding pipe 50 is a bent shape maintaining member. The biasing member is, for example, a push spring 60.

In the present embodiment, for easy distinction between end portions of these members, in the following description, an end portion on the distal portion side which is the one end side of the bent posture holding portion 3 will be described as a first end portion 3a, and an end portion on the operation portion side which is the opposite side of the first end portion 3a will be described as a second end portion 3b. In addition, an end portion on the distal portion side of the over-tube 40 will be described as a third end portion 40a, and an end portion on the opposite side of the third end portion 40a will be described as a fourth end portion 40b. Besides, an end portion on the distal portion side of the bent posture holding pipe 50 will be described as a fifth end portion 50a, and an end portion on the opposite side of the fifth end portion 50a will be described as a sixth end portion 50b.

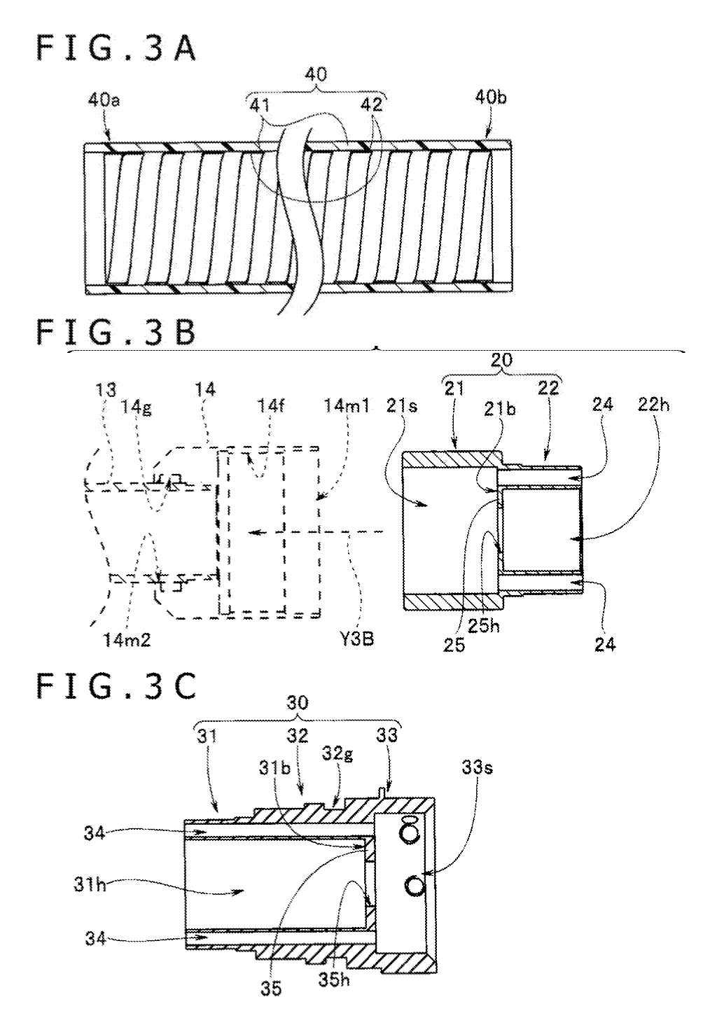

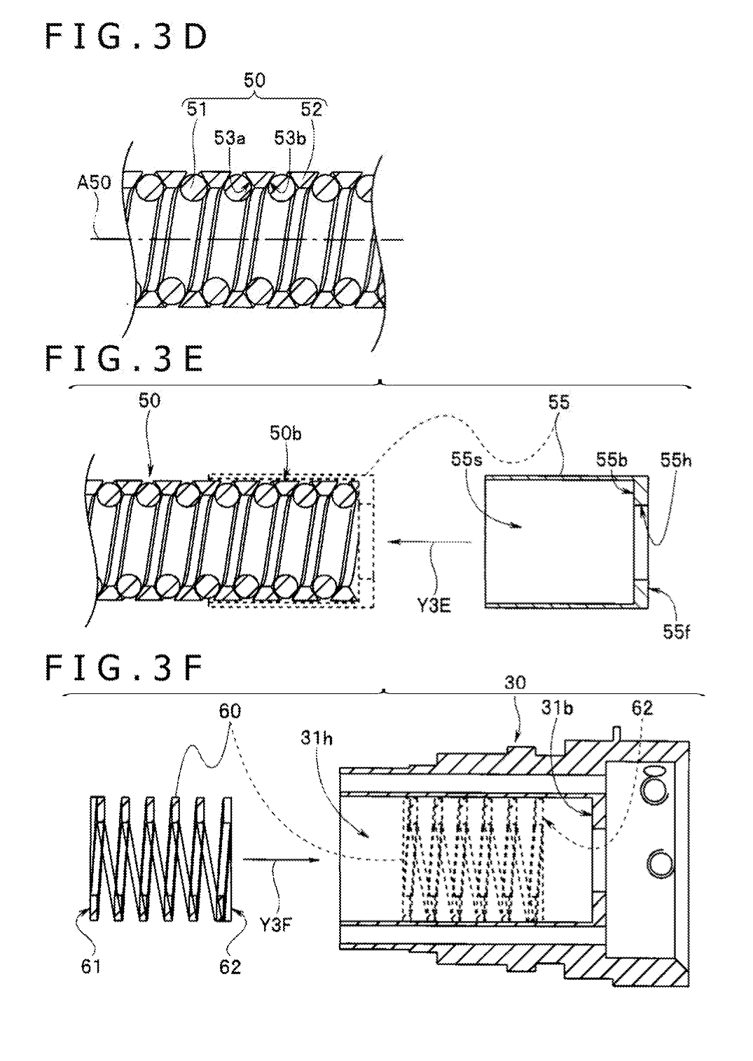

The over-tube 40 depicted in FIG. 3A functions both as a water-tight member and as a torsion preventing member, and includes a cover member 41 and a flex 42. The cover member 41 is a highly water-proofing and flexible member for securing a water-1 0 tight property, and is formed in an elongated thin pipe shape from a polymer member of, for example, fluororubber, silicone rubber or the like. On the other hand, the flex 42 is a spiral pipe formed by spirally winding a belt-like sheet made of a metal such as SUS. The flex 42 is integrally disposed on an inner peripheral surface of the cover member 41, and has such a degree of rigidity as not to be collapsed by a compressive force from the outer circumference side.

As depicted in FIG. 2, the over-tube 40 covers a range of the proximal side of the distal-side rigid portion 20 to the distal side of the proximal-side rigid portion 30, to thereby constitute mainly an exterior part of the bent posture holding portion 3. Note that the cover member 41 may be a heat-shrinkable tube which shrinks when heat is applied thereto. The distal-side rigid portion 20 depicted in FIG. 3B constitutes the first end portion 3a, which is the end portion on the distal side of the bent posture holding portion 3. The distal-side rigid portion 20 is a stepped pipe, including mainly a distal-side large-diameter portion 21 and a proximal-side small-diameter portion 22.

On an outer peripheral surface of the proximal-side small-diameter portion 22, an inner peripheral surface of the third end portion 40a of the over-tube 40 is disposed. The third end portion 40a of the over-tube 40 is integrally attached to the proximal-side small-diameter portion 22 by adhesion. The proximal-side small-diameter portion 22 can be wrapped with thread, then the third end portion 40a can be integrally attached with adhesion on the thread. An insertion portion side base 14 includes a distal-side rigid portion mounting port 14m1 and a rigid pipe mounting port 14m2 depicted in broken line in FIGS. 2 and 3B. The distal-side large-diameter portion 21 is inserted into the distal-side rigid portion mounting port 14m1 as indicated by arrow Y3B in FIG. 3B, and is thereafter integrally attached by adhesion or bonding. The distal-side rigid portion 20 is secured to the distal-side rigid portion mounting port 14m1. A proximal portion of the rigid pipe 13 is secured to the rigid pipe mounting port 14m2 on the distal side of the insertion portion side base 14. The distal-side large-diameter portion 21 is disposed with a distal space portion 21s, which is a hole having a predetermined depth size and having an opening in an end face of the distal-side large-diameter portion 21. On the other hand, the proximal-side small-diameter portion 22 is disposed with a first hole 22h having a predetermined inside diameter and a predetermined depth and having an opening in an end face of the proximal-side small-diameter portion 22.

On the outer side of the first hole 22h, there are four distal-side bending wire holes 24 at intervals of, for example, 90 degrees in the circumferential direction. Center axis of the distal-side bending wire holes 24 are parallel to the center axis of the first hole 22h. The distal-side bending wire holes 24 each have openings in an end face of the proximal-side small-diameter portion 22 and a bottom surface 21b of the distal space portion 21s. A first partition wall 25 is set to a predetermined thickness. The first partition wall 25 separates the distal space portion 21s and the first hole 22h from each other. The inside of the distal space portion 21s and the inside of the first hole 22h thus are separated and communicate with each other through a first communication hole 25h.

Note that in the case where the bendable portion 12 is bent into two directions, two bending wire holes 24 may be disposed at intervals of 180 degrees in the circumferential direction of the first hole 22h. A first O ring 16a is disposed in an inner circumferential groove 14g in FIG. 2. The first O ring 16a is disposed in close contact with the outer peripheral surface of the rigid pipe 13, to thereby prevent a liquid from penetrating from the exterior into the inside of the bent posture holding portion 3. In addition, the distal-side large-diameter portion 21 may be able to make screw engagement with the distal-side rigid portion mounting port 14m1. In this case, the inner peripheral surface of the distal-side rigid portion mounting port 14m1 is formed with a female screw portion 14f, and the outer peripheral surface of the distal-side large-diameter portion 21 is formed with a male screw portion, not depicted, for screw engagement with the female screw portion 14f.

Besides, the insertion portion side base 14 may be integrally constituting a proximal portion of the proximal portion of the rigid pipe 13. In this instance, the inner circumferential groove 14g and the first O ring 16a are unnecessary. The proximal-side rigid portion 30 depicted in FIG. 3C constitutes the second end portion 3b on the other end side, namely, the side opposite to the first end portion 3a of the bent posture holding portion 3. The proximal-side rigid portion 30 is a stepped pipe, which includes mainly, a distal-side small-diameter portion 31, an intermediate portion 32, and a proximal-side large-diameter portion 33.

On an outer peripheral surface of the distal-side small-diameter portion 31, an inner peripheral surface of the fourth end portion 40b of the over tube 40 is disposed. Besides, the fourth end portion 40b of the over tube 40 is integrally attached to the distal-side small-diameter portion 31 by adhesion or yarn winding adhesion or the like. The distal-side small-diameter portion 31 is disposed with second holes 31h having a predetermined inside diameter and a predetermined depth and having an opening in an end face of the distal-side small-diameter portion 31. In the outer periphery of the second hole 31h, there are four proximal-side bending wire holes 34 in such a manner as to be opposed to the four distal-side bending wire holes 24 disposed in the distal-side rigid portion 20. The proximal-side bending wire holes 34 each have openings in an end face of the distal-side small-diameter portion 31 and a bottom surface 33b of a proximal space portion 33s.

A second partition wall 35 is set to a predetermined thickness. The second partition wall 35 separates the second hole 31h and the proximal space portion 33s from each other. The inside of the second hole 31h and the inside of the proximal space portion 33s are thus separated and communicate with each other through second communication hole 35h. Reference symbol 31b denotes a second hole bottom surface. The proximal-side large-diameter portion 33 is disposed with a proximal space portion 33s which has an opening in an end face of the proximal-side large-diameter portion 33. The proximal-side large-diameter portion 33 is disposed in an operation portion space of the operation portion 4 depicted in broken line in FIG. 2.

The intermediate portion 32 is disposed in a proximal-side rigid portion mounting port 15m which has an opening in a distal-side end face of the operation portion 4. A second O ring 16b in FIG. 2 is disposed in an outer circumferential groove 32g. The second O ring 16b is disposed in close contact with an inner peripheral surface of the proximal-side rigid portion mounting port 15m, thereby preventing a liquid from penetrating from the exterior into the inside of the operation portion 4.

Note that in the case where the bendable portion 12 is configured to be bent into two directions similar to the distal-side rigid portion 20, two proximal-side bending wire holes 34 may be disposed at intervals of 180 degrees in the circumferential direction of the second hole 31h. The bent posture holding pipe 50 depicted in FIG. 3D is a bent shape maintaining member, and is a pipe member which can be bent relative to the direction of a longitudinal axis A50 of the holding pipe 50 and maintains the bent shape.

The bent posture holding pipe 50 is a metallic pipe member including a coil spring 51 which is a first spiral pipe, and a wire material pipe 52 which is a second spiral pipe. The coil spring 51 is formed by spirally winding a wire material having a circular sectional shape, and has a predetermined resilient force. On the other hand, the wire material pipe 52 is formed by spirally winding, for example, a wire material having a trapezoidal sectional, and has a predetermined resilient force. The wire material pipe 52 has contact surfaces 53a and 53b, and is disposed in the outer-side periphery of the coil spring 51 so that the two contact surfaces 53a and 53b can make contact with the coil spring 51, whereby the bent posture holding pipe 50 is configured.

In the present embodiment, as depicted in FIG. 3E, a sliding tube 55 is secured at the sixth end portion 50b of the bent posture holding pipe 50. The sliding tube 55 includes an accommodation space 55s having a predetermined inside diameter and a predetermined depth size. The sliding tube 55 is fitted over the sixth end portion 50b of the bent posture holding pipe 50 as indicated by arrow Y3E. In other words, the sixth end portion 50b of the bent posture holding pipe 50 is inserted into the accommodation space 55s of the sliding tube 55.

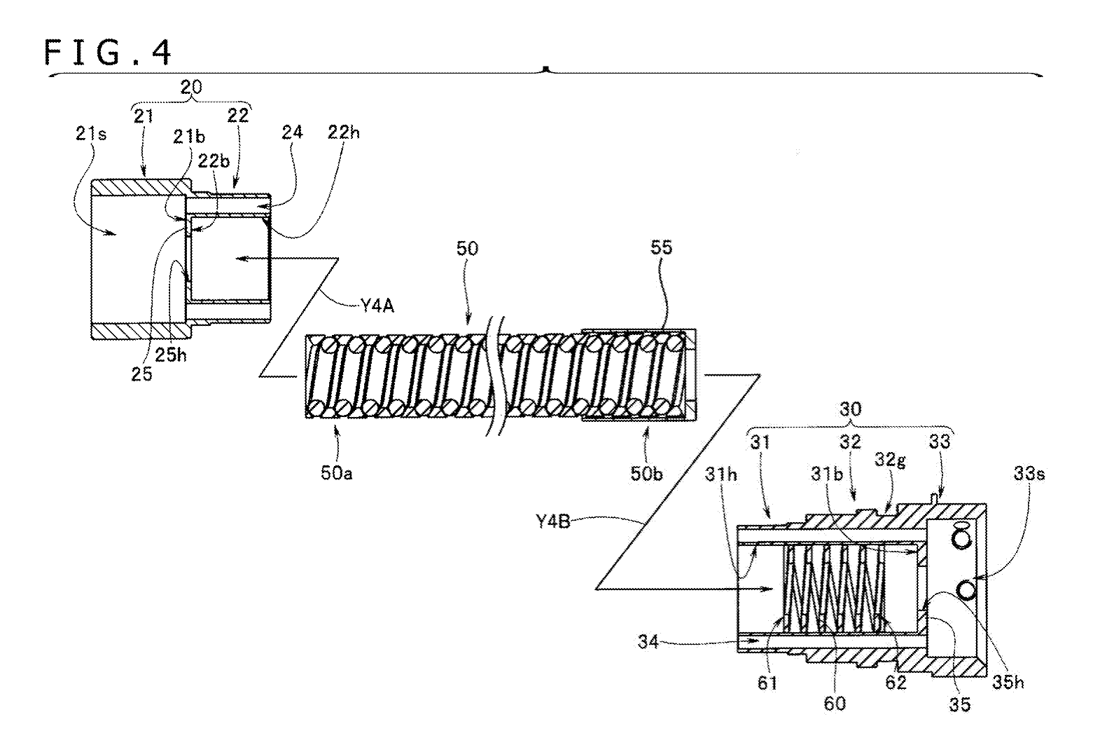

Besides, the sixth end portion 50b of the bent posture holding pipe 50 is integrally attached to the sliding tube 55 by, for example, adhesion in a state in which an end face of the sixth end portion 50b is in contact with an accommodation space bottom surface 55b, whereby the bent posture holding pipe 50 having the sliding tube 55 is configured, as depicted in FIG. 4. The exterior and the inside of the accommodation space 55s communicate with each other through a cylinder hole 55h. The cylinder hole 55h has openings in a cylinder end face 55f and the accommodation space bottom surface 55b.

FIG. 3F depicts the push spring 60, which is a coil spring having a predetermined resilient force, and is set to a predetermined length. The push spring 60 is a function holding member for maintaining a function of the bent posture holding pipe 50 as a bent shape maintaining member. Reference symbol 61 denotes a spring front surface, and reference symbol 62 denotes a spring rear surface. The push spring 60 is disposed in the second hole 31h of the proximal-side rigid portion 30 as indicated by arrow Y3F in FIG. 3F. Besides, in addition to the push spring 60, the sliding tube 55 is secured to the sixth end portion 50b as mentioned hereinbefore and is slidably disposed in the second hole 31h as depicted in FIG. 4. Specifically, the outside diameter of the sliding tube 55 and the outside diameter of the push spring 60 are substantially equal, and are smaller than the inside diameter of the second hole 31h.

Note that the outside diameter of the sliding tube 55 and the outside diameter of the push spring 60 may be different from each other. In addition, the push spring 60 is not limited to a coil spring, but may be a coned disc spring.

As depicted in FIG. 4, the fifth end portion 50a of the bent posture holding pipe 50 is inserted in the first hole 22h disposed in the distal-side rigid portion 20, as indicated by arrow Y4A. The bent posture holding pipe 50 is configured with the sliding tube 55 secured to the sixth end portion 50b. Besides, the fifth end portion 50a is integrally attached to the distal-side rigid portion 20 by adhesion, for example, in a state in which an end face of the fifth end portion 50a is in contact with a first hole bottom surface 22b.

On the other hand, the sixth end portion 50b of the bent posture holding pipe 50 is disposed in the second hole 31h disposed in the proximal-side rigid portion 30 in which the push spring 60 is preliminarily disposed, as indicated by arrow Y4B. In other words, the sliding tube 55 is secured to the sixth end portion 50 and is slidably disposed in the second hole 31h. Besides, the cylinder end face 55f of the sliding tube 55 makes contact with the spring front surface 61 of the expandable push spring 60 in the second hole 31h, to constantly keep the spring rear surface 62 in contact with the second hole bottom surface 31b. In other words, the push spring 60 is contracted as compared to its original length.

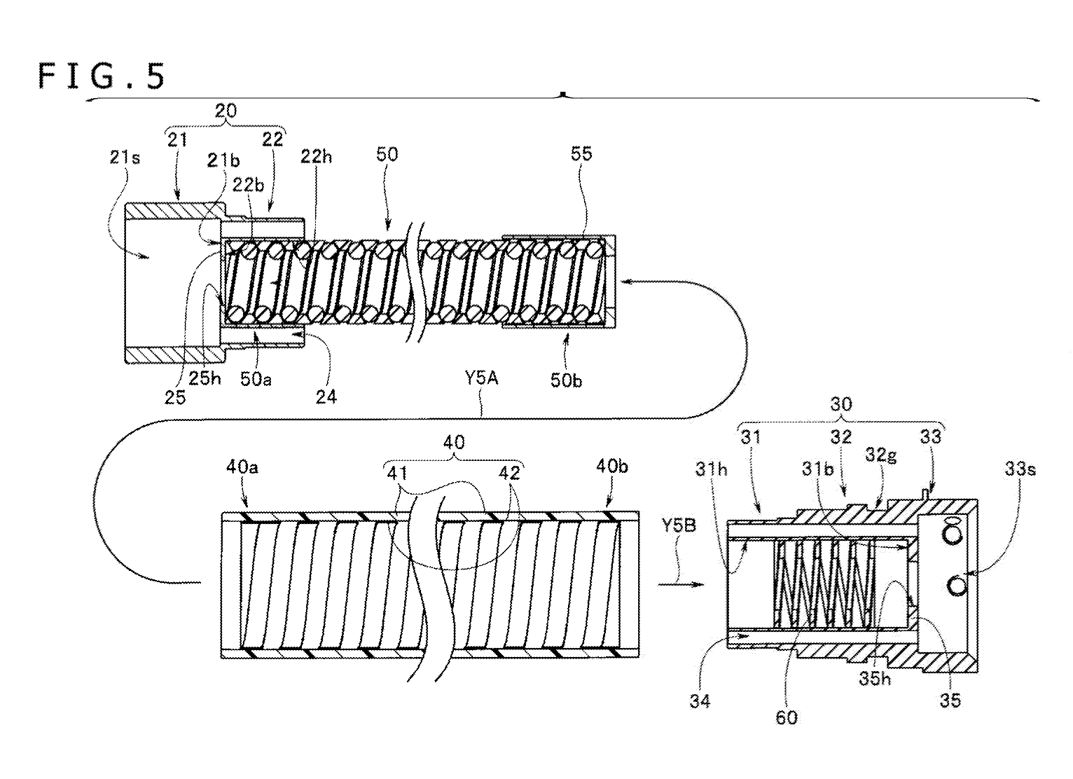

As depicted in FIG. 5, the third end portion 40a of the over-tube 40 is fitted over the outer periphery of the bent posture holding pipe 50 from the sixth end portion 50b side as indicated by arrow Y5A. The third end portion 40a is disposed on the outer peripheral surface of the proximal-side small-diameter portion 22 of the distal-side rigid portion 20 to which the fifth end portion 50a is secured. Then, the third end portion 40a is integrally attached to the distal-side rigid portion 20 by adhesion, for example.

On the other hand, the fourth end portion 40b of the over tube 40 is disposed on the outer peripheral surface of the distal-side small-diameter portion 31 of the proximal-side rigid portion 30 as indicated by arrow Y5B, and is thereafter integrally attached to the proximal-side rigid portion 30 by adhesion, for example. In other words, the bent posture holding pipe 50 is disposed in the pipe of the over-tube 40.

Note that in the case of attaching the third end portion 40a of the over-tube 40 to the distal-side rigid portion 20 and thereafter attaching the fourth end portion 40b of the over-tube 40 to the proximal-side rigid portion 30, the sliding tube 55 and the push spring 60 are preliminarily disposed in the second hole 31h beforehand. On the other hand, in the case of attaching the fourth end portion 40b of the over-tube 40 to the proximal-side rigid portion 30 and thereafter attaching the third end portion 40a of the over-tube 40 to the distal-side rigid portion 20, the fourth end portion 40b is attached to the proximal-side rigid portion 30, and, thereafter, the push spring 60 and the sliding tube 55 are sequentially disposed in the second hole 31h.

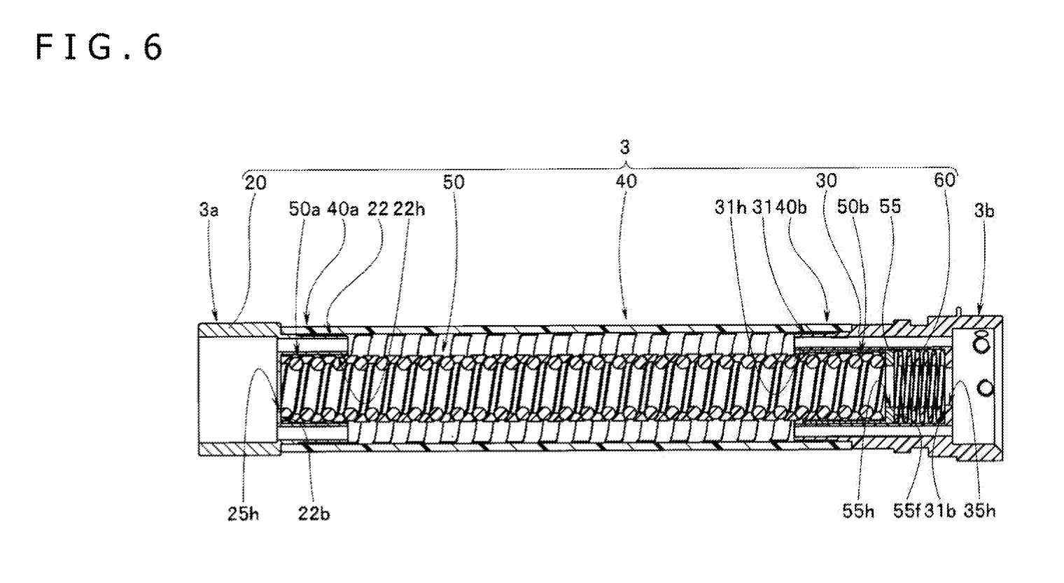

Then, as depicted in FIG. 6, the third end portion 40a of the over-tube 40 is attached to the distal-side rigid portion 20, and the fourth end portion 40b of the over-tube 40 is attached to the proximal-side rigid portion 30, whereby the bent posture holding portion 3 is assembled.

In this assembled state, the push spring 60 is disposed in the second hole 31h in a compressed state. In addition, the spring rear surface 62 of the push spring 60 is in contact with the second hole bottom surface 31b, and the spring front surface 61 is in contact with the cylinder end face 55f of the sliding tube 55. As a result, a biasing force from the compressed push spring 60 is constantly applied to the cylinder end face 55f of the sliding tube 55 secured to the sixth end portion 50b of the bent posture holding pipe 50. Consequently, the biasing force from the sixth end portion 50b toward the fifth end portion 50a along the longitudinal axis A50 is constantly exerted on the bent posture holding pipe 50.

The bent posture holding portion 3 configured in this way enables an angle of the insertion portion 2 relative to the operation portion 4 to be freely changed, and enables a bent state to be fixedly held by a shape holding force possessed by the bent posture holding pipe 50. When the bent posture holding portion 3 is repeatedly bent by a user, the coil spring 51 and the wire material pipe 52 constituting the bent posture holding pipe 50 rub against each other, to be worn. Then, the overall length of the bent posture holding pipe 50 is gradually shortened.

In the present embodiment, the fifth end portion 50a of the bent posture holding pipe 50 is attached in the first hole 22h, whereas the sixth end portion 50b is slidably disposed in the second hole 31h and is constantly biased by the push spring 60.

Therefore, as the overall length of the bent posture holding pipe 50 is gradually shortened as aforementioned, the overall length of the expandable push spring 60 is elongated, so that the total length of the bent posture holding pipe 50 and the push spring 60 is constant at all times. As a result, the function of the bent posture holding pipe 50 is maintained, and the problem of generation of loosening of the over-tube 40 is dissolved. Therefore, an increase in a torque exerted on the bent posture holding pipe 50 due to loosening of the over-tube 40 is prevented, and exertion of a partial load on the loosened portion of the over-tube 40, which might cause deterioration and breakage, can be prevented.

Note that in the aforementioned embodiment, the bent posture holding pipe 50 is configured by the coil spring 51 and the wire material pipe 52. However, the bent posture holding pipe 50 is configured such as to be able to maintain an arbitrarily bent state. Therefore, the bent posture holding pipe 50 may be an interlocked flexible tube formed by spirally winding a metallic belt material bent to be, for example, S-shaped in section.

In addition, the outside diameter of the proximal-side small-diameter portion 22 and the outside diameter of the distal-side small-diameter portion 31 may be set to the same size, and the inside diameter of the third end portion 40a and the inside diameter of the fourth end portion 40b of the over-tube 40 may be set to the same size, such that the third end portion 40a and the fourth end portion 40b can be disposed at either of the proximal-side small-diameter portion 22 and the distal-side small-diameter portion 31.

Besides, the sliding tube 55 may be secured not only to the sixth end portion 50b but also to the fifth end portion 50a. In this instance, the inside diameter of the first hole 22h is set to be greater than the outside diameter of the sliding tube 55. As a result, the fifth end portion 50a or the sixth end portion 50b with the sliding tube 55 secured thereto can be disposed in the first hole 22h.

In addition, according to this configuration, a configuration can be realized wherein the fifth end portion 50a and the push spring 60, or the sixth end portion 50b and the push spring 60, are slidably disposed in the first hole 22h. Further, there can also be realized, for example, a configuration wherein the fifth end portion 50a and the push spring 60 are slidably disposed in the first hole 22h, and the sixth end portion 50b and the push spring 60 are slidably disposed in the second hole 31h.

Besides, four operation wires, not depicted, for a bending operation of the bendable portion 12, an imaging cable 17 extended from an imaging unit, a light guide bundle 18 for transmitting illumination light, and the like are inserted in and passed through the insertion portion 2 depicted in FIG. 2. Of these members incorporated in the endoscope, the imaging cable 17 and the light guide bundle 18 are inserted in and passed through a flexible guide tube 19 in the bent posture holding portion 3, and the operation wires are inserted in and passed through a closely wound coil pipe, not depicted).

The closely wound coil pipe is inserted and passed between an outer peripheral surface of the bent posture holding pipe 50 and an inner peripheral surface of the over-tube 40, a coil pipe distal end portion is secured in the distal-side bending wire hole 24, and a coil pipe proximal end portion is secured in the proximal-side bending wire hole 34.

On the other hand, the guide tube 19 is inserted in and passed through the inside of the bent posture holding pipe 50, the inside of the push spring 60, and the inside of the cylinder hole 55h. A guide tube distal end portion is disposed in the distal space portion 21s, with a tube intermediate portion on the proximal side relative to a tube distal portion being attached to an inner surface of the first communication hole 25h by adhesion, for example. A guide tube proximal end portion is disposed in the proximal space portion 33s, with an intermediate portion on the distal side relative to a tube proximal portion being fixed to an inner surface of the second communication hole 35h by adhesion, for example.

In this way, the end portions of the closely wound coil pipe are secured individually in the distal-side bending wire hole 24 and the proximal-side bending wire hole 34, and the end portions of the guide tube 19 are attached by adhesion individually in the first communication hole 25h and to the second communication hole 35h, whereby metal debris generated due to rubbing of the coil spring 51 and the wire material pipe 52 against each other can be prevented from penetrating into the insertion portion 2 and the operation portion 4.

The present disclosure is not limited to the aforementioned embodiment, and various modifications can be performed without departing from the gist of the invention.

In addition, the insertion portion 2 including the bent posture holding portion 3 aforementioned is not limited in use to rigid endoscopes provided with a bendable portion for medical use, and may be used for bendable portion-lacking rigid endoscopes, industrial endoscopes, medical treatment devices, medical catheters and the like.

In sum, one aspect of the disclosed technology is directed to a flexible tube for an endoscope comprises a tube formed into an elongated thin pipe shape. A first rigid portion is attached to one end side of the tube. A second rigid portion is attached to a second end portion opposite to the one end side of the tube. The bent shape maintaining member is formed in a pipe shape from a metallic member and is disposed in a pipe of the tube. The bent shape maintaining member is capable to be bent relative to a direction of a longitudinal axis and to maintain a bent shape. One end side of the bent shape maintaining member is attached to the first rigid portion and an opposed end of the bent shape maintaining member is slidably disposed at the second rigid portion. A biasing member that is disposed at the second rigid portion biases the opposed end portion of the bent shape maintaining member along the longitudinal axis of the bent shape maintaining member toward the one end side of the bent shape maintaining member.

The flexible tube is defined by an over-tube configures as an exterior part that functions also as a water-tight member for maintaining the inside thereof in a water-tight state. The first rigid portion is a rigid pipe of an insertion portion of the endoscope and the second rigid portion is an operation portion of the endoscope. The bent shape maintaining member includes a first spiral pipe formed by winding in a spiral shape and a second spiral pipe that is disposed to make contact with the first spiral pipe at an outer periphery of the first spiral pipe and is formed by winding in a spiral shape. The first rigid portion has a first hole and the second rigid portion has a second hole.

Another aspect of the disclosed technology is directed to an endoscope comprises a flexible tube being defined by a tube having respective opposed first and second ends and being formed into an elongated thin pipe shape. A first rigid portion is attached to the first end of the tube. A second rigid portion is attached to the second end of the tube. A bent shape maintaining member having respective opposed third and fourth ends and are disposed in the elongated thin pipe shape of the tube and are capable to be bent relative to a direction of a longitudinal axis of the bent shape maintaining member and are maintained in a bent shape with the third end is attached to the first rigid portion and the fourth end is slidably disposed at the second rigid portion. A biasing member is disposed at the second rigid portion that biases the fourth end along the longitudinal axis of the bent shape maintaining member toward the third end.

The tube is defined by an over-tube that configures an exterior part functions as a water-tight member for maintaining the inside thereof in a water-tight state. The first rigid portion is defined by a rigid pipe of an insertion portion of the endoscope. The second rigid portion is defined by an operation portion of the endoscope. The bent shape maintaining member includes a first spiral pipe formed by winding in a spiral shape. A second spiral pipe is disposed to make contact with an outer periphery of the first spiral pipe and is formed by winding in a spiral shape. The respective first and second rigid portions include respective first and second holes.

A further aspect of the disclosed technology is directed to an endoscope comprises a flexible tube defined by a tube having respective opposed first and second ends and formed into an elongated thin pipe shape. A first rigid portion is attached to the first end of the tube. A second rigid portion is attached to the second end of the tube. A bent shape maintaining member having respective opposed third and fourth ends and are disposed in the elongated thin pipe shape of the tube and capable to be bent relative to a direction of a longitudinal axis of the bent shape maintaining member and are maintained in a bent shape with the third end being attached to the first rigid portion and the fourth end being slidably disposed at the second rigid portion. A biasing member is disposed at the second rigid portion that biases the fourth end along the longitudinal axis of the bent shape maintaining member toward the third end so that the overall length of the bent shape maintaining member is gradually shortened and the overall length of biasing member is elongated, so that the total length of the bent shape maintaining member and the biasing member remains constant.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A flexible tube for an endoscope, comprising:
a tube formed into an elongated thin pipe shape;
a first rigid portion attached to one end side of the tube;
a second rigid portion attached to a second end portion opposite to the one end side of the tube;
a bent shape maintaining member that is formed in a pipe shape from a metallic member and is disposed in a pipe of the tube, being capable to be bent relative to a direction of a longitudinal axis and to maintain a bent shape, one end side of the bent shape maintaining member being attached to the first rigid portion, and an opposed end portion of the bent shape maintaining member being slidably disposed at the second rigid portion; and
a biasing member that is disposed at the second rigid portion biases the opposed end portion of the bent shape maintaining member along the longitudinal axis of the bent shape maintaining member toward the one end side of the bent shape maintaining member.

2. The flexible tube for an endoscope of claim 1, wherein the tube is defined by an over-tube that configures an exterior part that functions also as a water-tight member for maintaining the inside thereof in a water-tight state.

3. The flexible tube for an endoscope of claim 1, wherein the first rigid portion is a rigid pipe of an insertion portion of the endoscope, and the second rigid portion is an operation portion of the endoscope.

4. The flexible tube for an endoscope of claim 1,
wherein the bent shape maintaining member includes:
a first spiral pipe formed by winding in a spiral shape; and
a second spiral pipe disposed therein to make contact with the first spiral pipe at an outer periphery of the first spiral pipe and is formed by winding in a spiral shape.

5. The flexible tube for an endoscope of claim 1,
wherein the first rigid portion has a first hole, and
the second rigid portion has a second hole.

6. An endoscope comprising:
a flexible tube being defined by
a tube having respective opposed first and second ends and being formed into an elongated thin pipe shape;
a first rigid portion being attached to the first end of the tube;
a second rigid portion being attached to the second end of the tube;
a bent shape maintaining member having respective opposed third and fourth ends and being disposed in the elongated thin pipe shape of the tube and capable to be bent relative to a direction of a longitudinal axis of the bent shape maintaining member and being maintained in a bent shape with the third end being attached to the first rigid portion and the fourth end being slidably disposed at the second rigid portion; and
a biasing member being disposed at the second rigid portion that biases the fourth end along the longitudinal axis of the bent shape maintaining member toward the third end.

7. The endoscope of claim 6, wherein the tube is defined by an over-tube that configures an exterior part functions as a water-tight member for maintaining the inside thereof in a water-tight state.

8. The endoscope of claim 6, wherein the first rigid portion is defined by a rigid pipe of an insertion portion of the endoscope.

9. The endoscope of claim 6, wherein the second rigid portion is defined by an operation portion of the endoscope.

10. The endoscope of claim 6, wherein the bent shape maintaining member includes
a first spiral pipe formed by winding in a spiral shape; and
a second spiral pipe disposed so as to be able to make contact with an outer periphery of the first spiral pipe and formed by winding in a spiral shape.

11. The endoscope of claim 6, wherein the respective first and second rigid portions include respective first and second holes.

12. An endoscope comprising:
a flexible tube being defined by
a tube having respective opposed first and second ends and being formed into an elongated thin pipe shape;
a first rigid portion being attached to the first end of the tube;
a second rigid portion being attached to the second end of the tube;
a bent shape maintaining member having respective opposed third and fourth ends and being disposed in the elongated thin pipe shape of the tube and being able to be bent relative to a direction of a longitudinal axis of the bent shape maintaining member and being maintained in a bent shape with the third end being attached to the first rigid portion and the fourth end being slidably disposed at the second rigid portion; and
a biasing member being disposed at the second rigid portion that biases the fourth end along the longitudinal axis of the bent shape maintaining member toward the third end so that the overall length of the bent shape maintaining member is gradually shortened and the overall length of biasing member is elongated, so that the total length of the bent shape maintaining member and the biasing member remains constant.
